(19) United States
(12) Patent Application Publication
(10) Pub. No.: US 2022/0193675 A1
(43) Pub. Date: Jun. 23, 2022

(54) CELL DISPENSER

(71) Applicant: SOLENTIM LTD., Dorset (GB)

(72) Inventors: Aaron Figg, Dorset (GB); Claire Louise Richards, Dorset (GB)

(73) Assignee: SOLENTIM LTD., Dorset (GB)

(21) Appl. No.: 17/555,255

(22) Filed: Dec. 17, 2021

(30) Foreign Application Priority Data
Dec. 18, 2020 (GB) 2020097.8

(51) Int. Cl. B01L 3/00; C12N 1/04; C12N 5/071

(52) U.S. Cl. CPC B01L 3/502761; C12N 1/04; B01L 2300/0829; B01L 2200/0689; C12N 5/0602

(57) ABSTRACT

An apparatus is provided for dispensing cells which comprises a determination unit to perform a determination as to whether a receptacle (i.e. a well) contains exactly one sample cell. This is because a medical study may require each cell in a colony of cultivated sample cells to each be derived from a common sample cell. The apparatus also comprises a dispenser to dispense one or more feeder cells into the receptacle based on the determination (i.e. when it is determined that the receptacle contains exactly one sample cell). The feeder cells which the apparatus is arranged to dispense are one or more feeder cells which are adapted to be responsive to a trigger condition being met to trigger death of that feeder cell. When the trigger condition is met, such as by controlling an environment of the receptacle, cell death of the feeder cells in the receptacle is triggered which results in only sample cells remaining alive.

### RELATED APPLICATIONS

This application claims priority to Great Britain Patent Application Number GB 2020097.8, filed Dec. 18, 2020, the entirety of which is hereby incorporated by reference.

### TECHNICAL FIELD

The present technique relates to a dispensing method and apparatus. For example, the present technique may have relevance to the field of cell dispensing.

### BACKGROUND

It is often desirable to cultivate a single cell to allow it to multiply into a colony of cells which all derive from that single cell, for example during medical research such as for drug approval. Such a process usually involves providing a reservoir and using a pump together with a dispensing tube to dispense a sample into the reservoir. The size of each sample to be pumped into the reservoir is chosen such that, with some degree of probability, a sample well (reservoir) will contain a single cell. Each sample is then cultivated over a period of time. In this way, a number of cultivations take place and the results can be compared (e.g. averaged). However, this approach has a number of drawbacks. Firstly, there is no guarantee that any given sample will contain only a single sample cell. Those samples which do not have only a single sample cell will fail (e.g. if there are no sample cells dispensed) or be unusable (e.g. if more than one sample cell is dispensed). If the number of successful cultivations is too low, the entire process may be considered a failure and may have to begin again. This incurs additional costs and time delays. Indeed, it may not be possible to determine that multiple cells were dispensed in the first place which means that the entire batch of cultivated cells may need to be discarded. In addition, sample cells often do not cultivate without being within a growth medium such as among a colony of feeder cells. Hence, it is often necessary to modify the sample cells to enable them to multiply. However this can has disadvantageous consequences regarding the quality or suitability of the sample cells for certain medical studies. It is therefore desirable to improve the chances of performing cultivation on a single sample cell, while maintaining a given pharmacological standard of the cultivated sample cells.

### SUMMARY

Viewed from a first example configuration, there is provided an apparatus comprising a determination unit to perform a determination as to whether a receptacle contains exactly one sample cell; and a dispenser to dispense one or more feeder cells into the receptacle based on the determination, wherein each of the one or more feeder cells is adapted to be responsive to a trigger condition being met to trigger death of that feeder cell.

Viewed from a second example configuration, there is provided a method comprising the steps of: performing a determination as to whether a receptacle contains exactly one sample cell; and dispensing one or more feeder cells into the receptacle based on the determination, wherein each of the one or more feeder cells is adapted to be responsive to a trigger condition being met to trigger death of that feeder cell.

Viewed from a third example configuration, there is provided an apparatus comprising: means for performing a determination as to whether a receptacle contains exactly one sample cell; and means for dispensing one or more feeder cells into the receptacle based on the determination, wherein each of the one or more feeder cells is adapted to be responsive to a trigger condition being met to trigger death of that feeder cell.

### BRIEF DESCRIPTION OF THE DRAWINGS

The present invention will be described further, by way of example only, with reference to embodiments thereof as illustrated in the accompanying drawings, in which:

FIG. 1 illustrates an example apparatus in accordance with one embodiment;

FIG. 2 shows a receptacle after dispensing a first sample having zero cells and a second sample having one cell;

FIG. 3 shows a receptacle after dispensing a sample having multiple cells;

FIG. 4 shows a receptacle after dispensing a sample having one cell and a plurality of feeder cells;

FIG. 5 shows a receptacle after a sample cell and a feeder cell have multiplied during cultivation;

FIG. 6 shows a receptacle after feeder cells have died leaving only cells derived from a single sample cell;

FIG. 7 shows a flowchart illustrating a method of dispensing and cultivation in which exactly one sample cell is dispensed and in which cell death of dispensed feeder cells is triggered once a trigger condition is met;

FIG. 8 shows a flowchart illustrating a method of dispensing and cultivation in which exactly one sample cell is dispensed and in which a trigger condition is met by controlling one or more factors in response to determining that the sample cell has multiplied a predetermined number of times;

FIG. 9 shows a flowchart illustrating a method of dispensing and cultivation in which exactly one sample cell is dispensed and in which a trigger condition is met by controlling one or more factors in response to a timer expiring;

FIG. 10 shows a flowchart illustrating a method of dispensing and cultivation in which exactly one sample cell is dispensed and in which a trigger condition is met by controlling one or more factors in response to an amount of light exposure exceeding a threshold; and

FIG. 11 shows a flowchart illustrating a method of dispensing in which exactly one sample cell is dispensed into each of a plurality of wells.

### DESCRIPTION OF EMBODIMENTS

In accordance with one example configuration there is provided an apparatus comprising: a determination unit to perform a determination as to whether a receptacle contains exactly one sample cell; and a dispenser to dispense one or more feeder cells into the receptacle based on the determination, wherein each of the one or more feeder cells is adapted to be responsive to a trigger condition being met to trigger death of that feeder cell.

A sample, containing zero, one, or more than one sample cell is dispensed into a receptacle. By using the determination unit to determine whether or not the receptacle contains exactly one sample cell and then dispensing the feeder cells based on that determination it is possible to reduce or even eliminate the situation in which feeder cells are added to a receptacle which contains either zero sample cells or more than one sample cell. In some previous techniques, the sample cells have been modified in order to enable them to replicate without needing to be provided with feeder cells. However this can hinder the ability of sample cells to meet a given pharmacological standard which a given medical study may require, since the modification of the sample cells may affect the standard of the sample cells in an undesirable way. According to the present technique, feeder cells, which die in response to a trigger condition, can be added to a receptacle based on the determination of the number of sample cells which means that it is possible to provide improved conditions under which the sample cell may grow (i.e. replicate) to form a colony of sample cells all derived from the same sample cell. This means that the sample cell does not need to be modified in order to improve its ability to replicate. By adding feeder cells with a trigger condition to trigger the cell death of the feeder cells, it is possible to improve the growing conditions of the sample cell and then to trigger the cell death of the feeder cells leaving only a cultivated colony of sample cells remaining in the receptacle. Since the sample cells have not been modified in order to achieve growth, the results of the growth can be used in pharmacological research.

In some examples, the dispenser is adapted to dispense the one or more feeder cells when the determination unit determines that the receptacle contains exactly one sample cell. In many medical research studies, there is a need to produce a colony of sample cells which are all derived from a single sample cell (i.e. the original sample cell), for example, in order to improve the control of relevant variables in a given study. By determining that the receptacle contains exactly one sample cell, it is possible to determine that a cultivated colony of cells will be derived from one sample cell and any feeder cells which are dispensed into the receptacle. This means that it is possible to improve the reliability of colonies cultivated for medical research.

In some examples, the apparatus comprises a control unit to control one or more factors in order to cause the trigger condition. It is desirable to provide a mechanism for the removal of feeder cells at a particular point in time in order to leave only sample cells remaining in the receptacle. This is in order to improve the ease and reliability of performing medical research using the cultivated colony of cells. However, if the feeder cells are removed (i.e. die) too early, this can hinder the growth of the sample cells and may render the sample cells unsuitable to be used in medical research. Yet, if the feeder cells are removed too late, the medical study could take longer than is required or the sample cells may multiply more than is desired. Therefore, by controlling one or more factors in order to cause (e.g. activate) the trigger condition it is possible to control the point in time at which cell death of the feeder cells is triggered. It enables the trigger condition to trigger the death of the feeder cells in the receptacle at a particular point in time based on the requirements of a given medical study.

In some examples, the one or more factors comprise an environment of the receptacle. When conducting medical research studies, it is desirable to reduce the amount of interference with the colony of cells in a given receptacle. For example, using physical tools in order to remove feeder cells may contaminate the receptacle or damage the sample cells, thereby reducing the reliability of the sample cells for the purposes of a medical study. Therefore, it is desirable to provide a non-intrusive means of controlling the one or more factors in order to trigger cell death of the feeder cells. In these examples, the environment of the receptacle is able to be controlled by the control unit in order to activate the trigger condition to cause cell death of the feeder cells. This means that it is possible to trigger cell death of the feeder cells without needing to physically interfere with the contents of the receptacle.

In some examples, the control unit is adapted to control the one or more factors when the sample cell has multiplied a predetermined number of times. In some medical studies, there is a requirement for a sample cell to multiply a predetermined number of times. This may, for example, be to provide multiple copies of the same sample cell for which a measured attribute is averaged across each copy. It may be desirable to provide a specified number of samples, or to provide a number of samples wherein the number is within a specified range. By controlling the one or more factors (i.e. to trigger cell death of the feeder cells) when the sample cell has multiplied a predetermined number of times it is possible to improve the reliability of producing a cultivate colony of sample cells which is suitable for a given medical study.

In some examples, the determination unit comprises an image capture device to capture an image of the receptacle to determine whether the sample cell has multiplied the predetermined number of times. It is desirable to determine the number of times that the sample cell has multiplied, or to determine whether or not the sample cell has multiplied a predetermined number of times. However some techniques for determining this can be inaccurate. For example, it is possible to determine the number of times a sample cell has multiplied based on a duration over which the sample cell has been able to multiply in the receptacle. However, there are a number of factors which may affect the rate at which a sample cell is able to multiply. By using an image capture device to capture an image of the receptacle, an estimation of how many times the sample cell has multiplied can be improved. The image captured by the image capture device could be used to calibrate the time period based on the observed rate at which the sample cell multiplies. Alternatively (or additionally), the image capture device may capture an image of the receptacle (which contains the sample cells) and perform an image processing operation on the captured image in order to identify the number of sample cells in the receptacle. Some of the cells may be located on top of each other therefore a portion of the sample cells in the receptacle may be hidden from view of the image capture device. Therefore, in some examples, the determination unit may make an estimate of the total number of sample cells in the receptacle based on the number of sample cells visible to the image capture device. Accordingly, it is possible to more accurately determine the number of cells in the receptacle.

In some examples, the control unit is adapted to control the one or more factors after a predetermined period. For some sample cells, the growth rate of sample cells can be consistent. For example, the sample cell may be known to multiply once over a given multiplication period. Therefore, it is possible to determine (estimate) the number of sample cells in a receptacle based on the period of time which has elapsed since the original sample cell was dispensed into the receptacle. Alternatively, some medical studies may have a requirement that the sample cells be allowed to multiply in the receptacle for a given amount of time. For example, to test the growth rate for a given type of sample cell, or a given number of sample cells of the same type. By controlling the one or more factors to trigger the cell death of the feeder cells after a predetermined period, it is possible to more easily control the point at which the sample cells are revealed, by removing the feeder cells, in accordance with the requirements of the medical study.

In some examples, the environment comprises a light exposure. By controlling the triggering of cell death of the feeder cells based on light exposure, it is possible to accurately (e.g. almost instantly) control the point in time at which the trigger condition is achieved and thereby the point in time at which cell death is triggered. This can help to improve the accuracy of sample cells which are cultivated for a medical study because it is easier to produce sample cells which meet the particular sample cell growth requirements of a given medical study. In particular, the trigger is almost instantly reached for each feeder cell at effectively the same time.

In some examples, the sample cell is resilient to the environment. The purpose of dispensing at least one feeder cell into the receptacle is to improve the growing conditions of the sample cell to enable it to more easily multiply into a colony of sample cells. However, for the purposes of many medical studies it is undesirable for the feeder cells to remain present in the receptacle once the colony of sample cells has been cultivated. However, it is important that the sample cells are not significantly affected by the trigger condition being met, otherwise, the sample could be ruined. Therefore, by providing sample cells which are resilient to the environment that triggers cell death of the feeder cells, it is possible to prevent the sample cells from also being killed off, and therefore the quality of the sample can be improved.

In some examples, the trigger condition is that the feeder cell has lived for a predetermined lifetime. The feeder cells are provided to improve the conditions for the dispensed sample cell to grow. However, as previously discussed, the feeder cells are removed (i.e. killed off) when desired in order to leave only the sample cells remaining in the receptacle. This may be achieved by configuring the feeder cells to die after a predetermined period of time (lifetime). This means that it is possible to program the cells to trigger the condition without requiring any external equipment or trigger, such as changing the amount of light exposure or temperature of the environment of the receptacle. This can have the benefit of simplify the triggering of feeder cell death.

In some examples, the apparatus comprises a feeder cell reservoir to store feeder cells to be dispensed by the dispenser. The apparatus may be used multiple times to dispense respective sample cells into a large number of reservoirs. This may be in order to provide a set of sample cell colonies to be used in a medical study in order for a corresponding set of data to be averaged. Therefore, by providing a feeder cell reservoir to store feeder cells it is possible to produce a large number of feeder cells to be maintained in the reservoir. This means that they can be continually dispensed into successive receptacles. Hence, the rate at which receptacles can be populated with a sample cell can be increased.

In some examples, the determination unit comprises an image capture device to capture an image of the sample in the receptacle to determine whether the receptacle contains only one sample cell. It can be important to determine whether the receptacle initially contains only one sample cell. If zero sample cells are provided then no growth can occur. Meanwhile, if more than one sample cells are provided then multiplication may originate from two different cells leading to the grown sample containing cells of two different types and this typically invalidates many pharmacological studies. However, it can be difficult to determine whether or not exactly one sample cell has been dispensed into the receptacle. For example, the size of the sample to be dispensed by the sample may be controlled so as to make it more likely that exactly one sample cell will be dispensed; however this approach leads to a considerable number of errors. By providing an image capture device to capture an image of the sample in the receptacle, it is possible to determine, with improved accuracy, whether or not the receptacle contains only one sample cell. The image capture device may, for example, perform edge detection on the captured image data to determine if an edge of a single cell can be detected. Therefore, the reliability of determining whether or not only a single cell is contained in a receptacle can be improved.

In some examples, the dispenser is adapted to dispense the sample cell into the receptacle. By dispensing the sample cell into the receptacle, as well as the at least one feeder cell, the dispenser can be re-used and hence the cost of production and the overall size of the apparatus can be reduced. While there may be separate parts of the dispenser which relate to the dispensing of the sample cell as opposed to the at least one feeder cell, the overall dispenser may share at least some components, such as a pump, power supply or dispensing tube, which enable these benefits to be achieved.

In some examples, the dispenser is adapted to dispense the sample cell into the receptacle when the determination unit determines that the receptacle contains exactly zero sample cells. It is desirable to provide a receptacle which contains exactly one sample cell, as discussed above, which means that typically a single sample cell may be dispensed into a given receptacle and then a subsequent determination process may take place to determine whether or not the receptacle does indeed contain only a single sample cell. However, in some cases, it may be that there is already a sample cell in the receptacle. In such an event, dispensing a further sample cell into the receptacle would result in the receptacle containing more than one sample cell and hence the receptacle would be rejected for use within certain medical studies. By dispensing the sample cell when it is determined that the receptacle contains zero sample cells, it is possible to reduce occurrences in which a receptacle contains zero sample cells.

In some examples, the apparatus comprises a storage unit to store an indicator that the receptacle contains more than one sample cell when the determination unit determines that the receptacle contains more than one sample cell. This indicator may be used to indicate that a corresponding receptacle, which contains more than one sample cell, should not be used as part of the medical study since it would generate erroneous results and decrease the accuracy of the findings of the medical study. Therefore, by providing a storage unit to store an indicator to indicate that the receptacle contains more than one sample cell when it is determined as such, it is possible to improve the accuracy of a medical study by preventing erroneous samples from being used.

In some examples, the sample cell is a human tissue cell. By providing human tissue cells, such as stem cells, as the sample cell to be dispensed by the dispenser, it is possible to perform medical studies on the sample cell. Various types of human tissue cells may multiply at different rates or demonstrate different characteristics as they multiply into a colony of sample cells. Therefore, by dispensing a single human tissue cell as the sample cell into a receptacle, it is possible to perform a wider range of medical studies in relation to the behaviour of human tissue.

Some particular embodiments will now be described with reference to the figures.

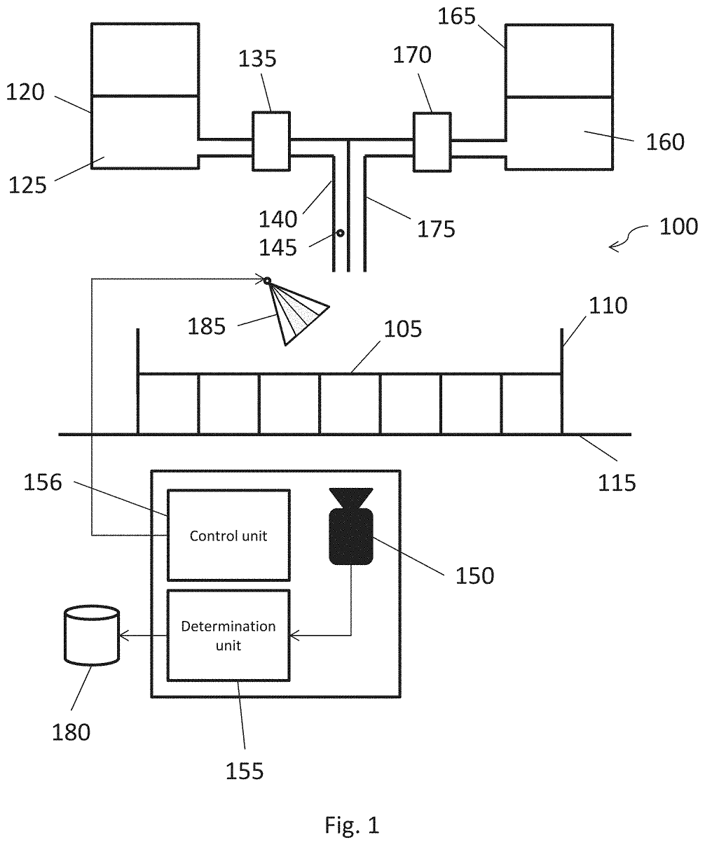

FIG. 1 illustrates an example apparatus in accordance with one embodiment.

The apparatus 100 includes a well 105 (an example of a receptacle), which forms part of a microtiter well plate 110 together with a plurality of other wells. The well 105 is able to move along a track 115 by virtue of the microtiter well plate moving along the track 115, the microtiter well plate being placed on a carriage connected to an actuator such as a screw turned by a motor controlled by a control system. A reservoir 120 contains a mixture 125 comprising a number of sample cells mixed together with a growth medium. A pump 135 is provided to extract a small quantity of the mixture 125 (a sample) from the reservoir and to dispense the sample through a tube 140 into a well 105. In this embodiment, the well 105 is located beneath the tube 140. The tube 140 is such that it is just wide enough to pass one of the cells. Accordingly, for a given concentration of cells in the mixture 125 and for a given sample size, there is a probability with which a sample will contain a single cell 145. In this example, the pump 135 and tube 140 collectively make up a dispenser. An image capture device 150 captures an image of the sample in the well 105 once the sample has been dispensed into the well 105. A determination unit 155 processes the image and determines, through image analysis, whether the sample or samples in the well 105 contain zero cells, one cell, or more than one cell. The action that is subsequently taken depends on which of these three conditions is met.

In the event that zero cells are detected, the well 105 and the tube 140 are moved relative to one another while still keeping a position such that a subsequent sample will be dispensed into the well 105. In other words, the well 105 and the tube 140 are moved relative to each other such that a subsequent sample will be dispensed into a different part of the well 105. In this example, the relative movement occurs by the microtiter well plate 110 being moved slightly along the track 115.

In the event that one cell is detected, feeder cells 160 stored in a secondary reservoir 165 are provided by using a secondary pump 170 and a secondary dispensing tube 175. These feeder cells are provided so as to be responsive to a trigger condition to trigger cell death of the feeder cells. After the death of such feeder cells, the sample cells derived from the single sample cell dispensed into the well 105 are the only cells which remain alive in the well 105. Sufficient feeder cells are provided to the well 105 in order to encourage cultivation of cells whilst not over-filling the well 105. By providing the majority of the growth medium 160 after it has been established that the well 105 includes a single cell, it is possible to improve the likelihood that the sample cell will be usable for a given medical study requiring the sample cells of the cultivated colony of sample cells to be derived from a single original sample cell. Once the feeder calls 160 have been dispensed, the microtiter well plate 110 is moved so that a subsequent sample will be dispensed into an unused well, i.e. a well that has not had any samples dispensed into it during the process. Furthermore, the image of the sample in the well 105 is stored in a storage medium 180 for later retrieval by the user.

In the event that more than one cell is detected, an error action is performed. In this embodiment, the error action includes making note of the particular well 105 into which the sample was dispensed. For example a number or other identifier that uniquely identifies the well 105 in the microtiter well plate 110 is made. At the end of the overall process, the user is informed of those wells that were marked. In this embodiment, the image of the well 105 having more than one cell is stored in a storage medium 180 for later retrieval by the user.

In any of the above cases, unless the last well has been filled, the process is repeated, with another sample being dispensed.

As a consequence of the above, it is possible to reduce the number of occasions in which a well of cultivated sample cells cannot be used. In addition, the scenario of a sample containing zero cells can be easily corrected for by dispensing further samples and the scenario of a sample containing more than one cell (which may not be easily corrected) will occur more rarely.

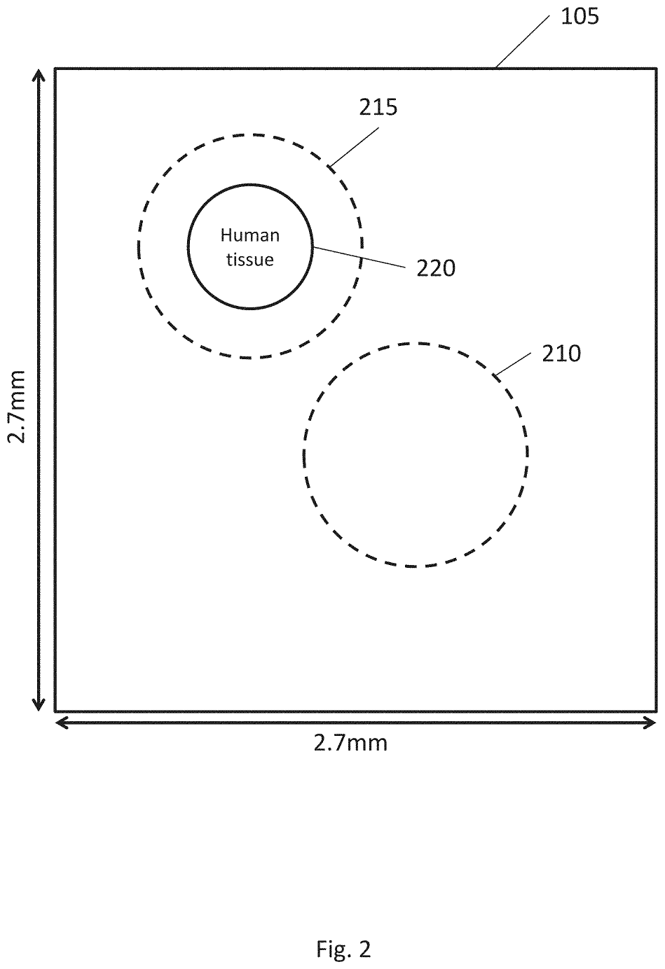

FIG. 2 shows a receptacle, e.g. well 105 after dispensing a first sample 210 having zero sample cells and a second sample 215 (which may, for example, be a human tissue cell) having one sample cell 220. In this example, the well 105 has an area of 2.7 mm by 2.7 mm and a volume of 110 microlitres. After the first sample 210 is dispensed, the determination unit 155 determines that the sample in the well 105 contains no sample cell. Consequently, the dispenser and the well 105 are moved relative to each other such that a subsequent sample 210 will be dispensed in a different part of the well 105. When the second sample 210 is dispensed, the determination unit 155 again processes an image of the well 105 and determines that the well 105 contains exactly one sample cell 215. Accordingly, at least one feeder cell from among the feeder cells 160 in the feeder cell reservoir 165 can be provided into the well 105. Additionally, the image of the well 110 containing a single cell is output. For example, the image can be output to a storage medium 180 or can be printed on a printer. Having dispensed the feeder cells, the microtiter well plate 110 is moved such that further samples will be dispensed into a different well.

Alternatively, if the current well 105 is the last well in the microtiter well plate 110 then the process stops.

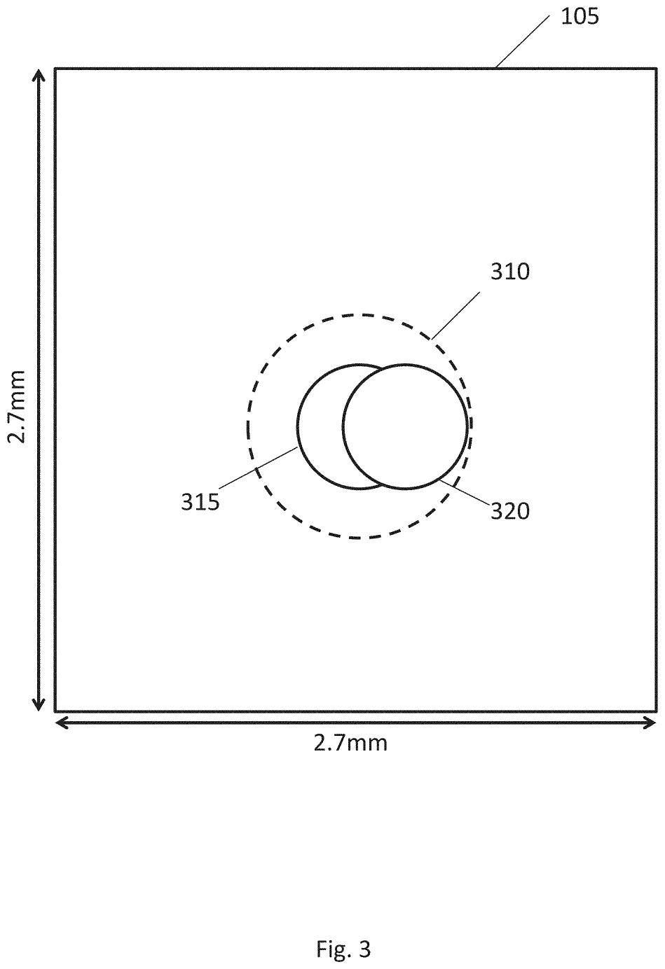

FIG. 3 shows a receptacle, e.g. well 105 after dispensing a sample 310 having two cells 315, 320. In this example, again, the well 105 has an area of 2.7 mm by 2.7 mm and a volume of 110 microlitres. After the first sample 305 is dispensed, the determination unit 155 processes an image of the well 105 and determines that the well 105 contains more than one cell (e.g. two cells 315, 320). Although the two cells 315, 320 slightly overlap each other, the determination unit 155 determines that there is not only one cell in the well 105. The well 105 is marked. For example, the processing circuitry 105 can immediately inform the user or alternatively can keep track of an ID number of the particular well and inform the user at the end of the process that the well should be disregarded due to having more than one cell. In any event, unless this is the final well in the microtiter well plate 110, the microtiter well plate is moved such that subsequent samples are dispensed into a different well.

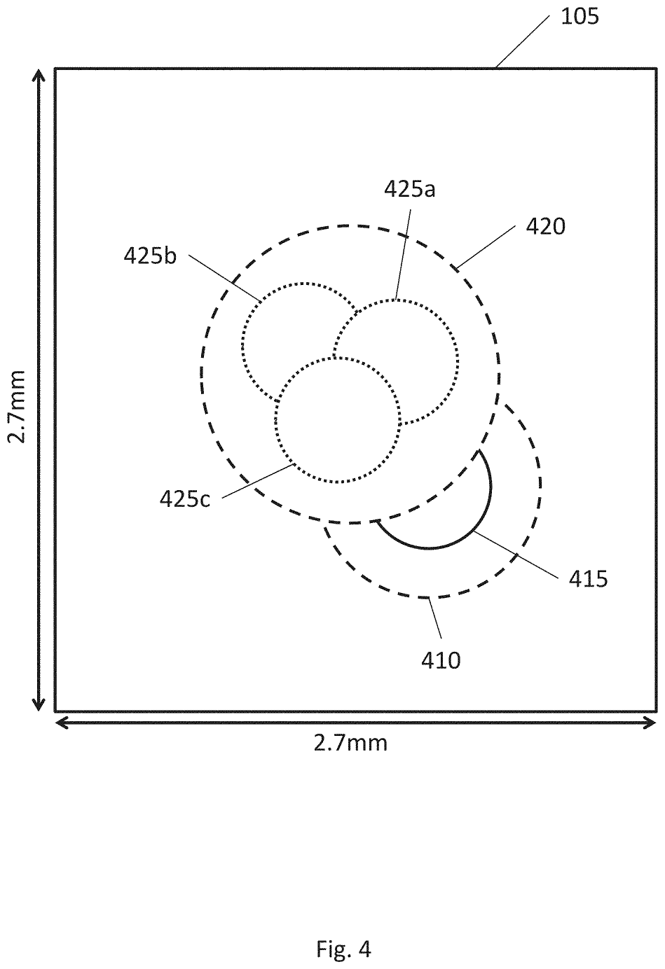

FIG. 4 shows a receptacle, e.g. well 105 after dispensing a sample 410 having one sample cell 415. In this example, the well 105 also has an area of 2.7 mm by 2.7 mm and a volume of 110 microlitres. After the sample 410 is dispensed, the determination unit 155 determines that the sample in the well 105 contains exactly one sample cell 415. Accordingly, at least one feeder cell 425a, 425b, 425c from among the feeder cells 160 in the feeder cell reservoir 165 can be provided into the well 105. The at least one feeder cell 425a, 425b, 425c dispensed into the receptacle is adapted to be responsive to a trigger condition being met to trigger cell death of that feeder cell 425a, 425b, 425c. For example, the trigger may be one or more factors, such as an environment of the receptacle. Thus, the sample cell 415 is provided with at least one feeder cell 425a, 425b, 425c to improve the growing conditions of the sample cell 415, to enable it to be cultivated into a colony of sample cells, however the feeder cells 425a, 425b, 425c can be killed off when required in order to leave only sample cells derived from the original sample cell 415 remaining alive in the receptacle 105. The image of the well 105 containing a single cell is output. For example, the image can be output to a storage medium 180 or can be printed on a printer. The microtiter well plate 110 is also moved such that further samples will be dispensed into a different well. Alternatively, if the current well 105 is the last well in the microtiter well plate 110 then the process stops.

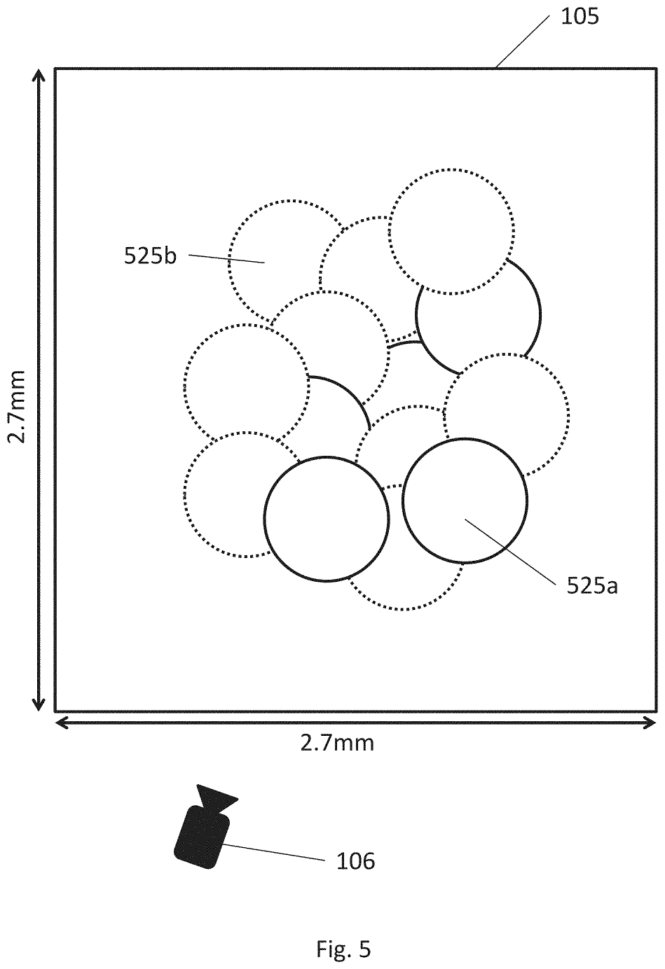

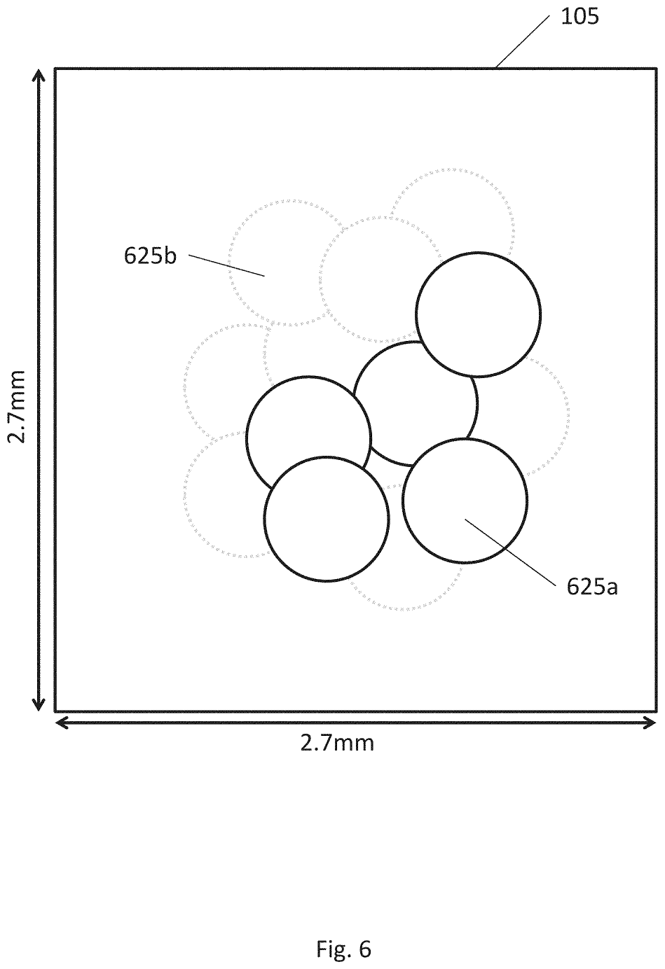

FIG. 5 shows a receptacle, e.g. well 105, after a sample cell 415 has multiplied during the process of cultivation. In this example, the well 105 also has an area of 2.7 mm by 2.7 mm and a volume of 110 microlitres. At the point represented in FIG. 5, the sample cell 415 has multiplied several times to produce a colony of sample cells 525a. In addition, in this example, the feeder cells have also multiplied to form a colony of feeder cells 525b. The feeder cells provide a more effective growing environment for the sample cells 525a to multiply. An image capture device 106 is also illustrated which captures an image of the well 105 to determine whether the sample cell has multiplied the predetermined number of times. If it has, then it controls the one or more factors to cause the trigger condition to trigger feeder cell death, as illustrated in FIG. 6.

FIG. 6 shows a receptacle, e.g. well 105 after a sample cell 415 has multiplied during the process of cultivation and after which the feeder cells have died (e.g. as a result of the image capture device capturing an image used to determine that the sample cell has multiplied the predetermined number of times). In this example, the well 105 also has an area of 2.7 mm by 2.7 mm and a volume of 110 microlitres. At the point represented in FIG. 6, a trigger condition to trigger the cell death of the feeder cells has been activated such that the feeder cells are no longer alive. The trigger condition may be based on one or more factors, such as the environment of the receptacle or the expiry of a predetermined amount of time since the feeder cells were created. In some examples, the environment may be changed, such as by being exposed to an increased amount of light, which may cause the triggering of the feeder cell death. As shown in FIG. 6, the sample cells are not killed by the environment, but rather are resilient to the environment, as shown by the sample cells remaining after the feeder cells have died. However, there are a number of ways in which the cell death of the feeder cells may be triggered. Once the feeder cells have died, they may, for example, begin to decay and then be able to be easily washed away and removed from the receptacle. In any case, the death of the feeder cells means that a given medical study to be performed in respect of the colony of sample cells can be performed more easily. The feeder cells, could take the form of fibroblasts such as MEF's (mouse embryonic fibroblasts) which have been gene edited to express a light-responsive protein (such as KillerRed, supplied by Evrogen). Upon activation by light, the protein generates reactive oxygen species (ROS) that damage neighbouring molecules and ultimately results in cell death. These cells can be mitotically inactivated prior to seeding and dispensed in the media following single cell deposition. The feeder cells would adhere and support the growth of the single cell of interest but they themselves would not divide due to being mitotically inactive. Once the colony of desired cells had achieved a size that no longer requires support from feeder cells, the feeder cells could be destroyed by light activation of KillerRed.

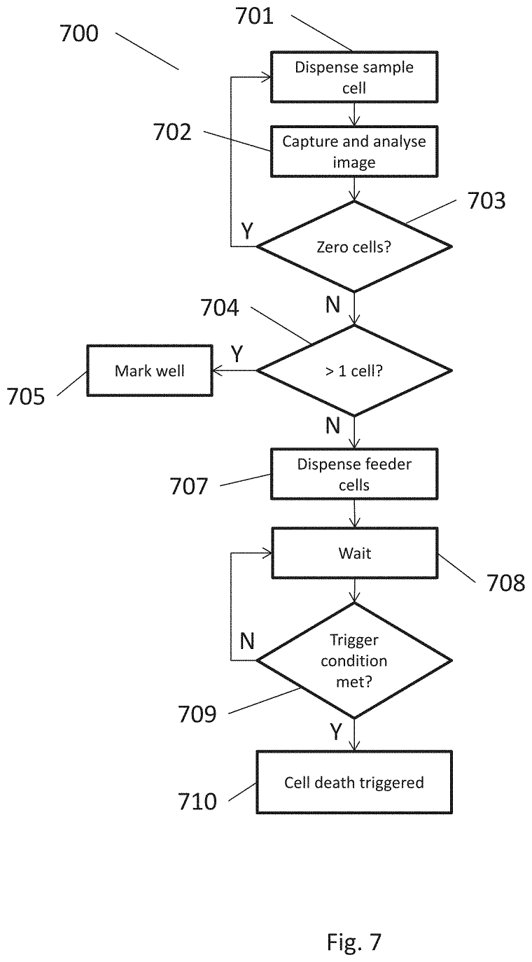

FIG. 7 illustrates a flow chart 700 that shows a method of dispensing. In step 701, a sample is dispensed into the current well 105. In step 702, an image of the sample cell in the well 105 is captured and analysed. In step 703 it is determined whether the receptacle (current well) 105 contains exactly zero cells, on the basis of the analysis of the captured image. If it does contain zero cells then the method returns to step 701 to dispense a further sample cell into the well 105. However, if the well 105 contains more than zero sample cells, the method proceeds to step 704 where it is determined whether the well contains more than one sample cell. If the well 105 does contain more than one sample cell, then the well is marked at step 705 and an indication of the well may be stored in the storage unit 180. However, if the well 105 does not contain more than one sample cell it is concluded that the well 105 contains exactly one sample cell, therefore the method can proceed to step 707 at which point at least one feeder cell is dispensed into the well 105 along with the sample cell. Then, at step 708 the method waits (pauses) and at step 709 it is determined whether or not the trigger condition to trigger cell death of the feeder cells is met. If the trigger condition is not met, the method returns to wait at step 708, however if the trigger condition is met, then the method proceeds to step 710 at which point cell death of the feeder cells is triggered. In other words, steps 708 and 709 achieve the effect of waiting until the trigger condition is met. When the trigger condition is met, step 710 causes each of the feeder cells to be killed in order to leave only sample cells derived from the original sample cell remaining alive in the well 105.

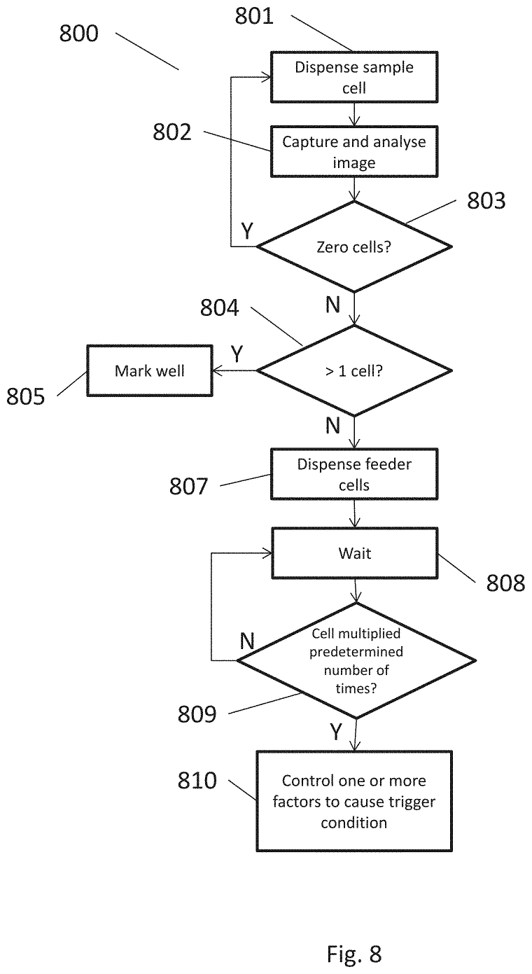

FIG. 8 illustrates a flow chart 800 that shows a method of dispensing. In step 801, a sample is dispensed into the current well 105. In step 802, an image of the sample cell in the well 105 is captured and analysed. In step 803 it is determined whether the receptacle (current well) 105 contains exactly zero cells, on the basis of the analysis of the captured image. If it does contain zero cells then the method returns to step 801 to dispense a further sample cell into the well 105. However, if the well 105 contains more than zero sample cells, the method proceeds to step 804 where it is determined whether the well contains more than one sample cell. If the well 105 does contain more than one sample cell, then the well is marked at step 805 and an indication of the well may be stored in the storage unit 180. However, if the well 105 does not contain more than one sample cell it is concluded that the well 105 contains exactly one sample cell, therefore the method can proceed to step 807 at which point at least one feeder cell is dispensed into the well 105 along with the sample cell. Then, at step 808 the method waits (pauses) and at step 809 it is determined whether or not the sample cell has multiplied the predetermined number of times. If the sample cell has multiplied the predetermined number of times, the method returns to wait at step 808, however if the sample cell has not yet multiplied the predetermined number of times, then the method proceeds to step 810 at which point one or more factors are controlled by the control unit 156 in order to cause the trigger condition to be met to trigger cell death of the feeder cells in the well 105. In other words, steps 808 and 809 achieve the effect of waiting until the sample cell has multiplied the predetermined number of times. When the sample cell has multiplied the predetermined number of times, step 810 causes each of the feeder cells to be killed by controlling the one or more factors to cause the trigger condition, in order to leave only sample cells derived from the original sample cell remaining alive in the well 105.

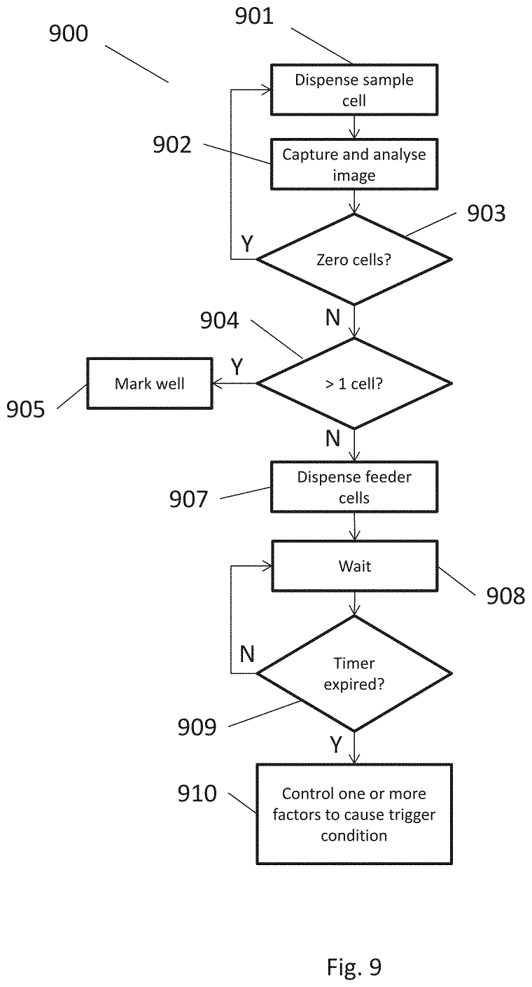

FIG. 9 illustrates a flow chart 900 that shows a method of dispensing. In step 901, a sample is dispensed into the current well 105. In step 902, an image of the sample cell in the well 105 is captured and analysed. In step 903 it is determined whether the receptacle (current well) 105 contains exactly zero cells, on the basis of the analysis of the captured image. If it does contain zero cells then the method returns to step 901 to dispense a further sample cell into the well 105. However, if the well 105 contains more than zero sample cells, the method proceeds to step 904 where it is determined whether the well contains more than one sample cell. If the well 105 does contain more than one sample cell, then the well is marked at step 905 and an indication of the well may be stored in the storage unit 180. However, if the well 105 does not contain more than one sample cell it is concluded that the well 105 contains exactly one sample cell, therefore the method can proceed to step 907 at which point at least one feeder cell is dispensed into the well 105 along with the sample cell. Then, at step 908 the method waits (pauses) and at step 909 it is determined whether or not the timer has expired to trigger cell death of the feeder cells. If the timer has not expired, the method returns to wait at step 908, however if the timer has expired, then the method proceeds to step 910 at which point one or more factors are controlled by the control unit 156 in order to cause the trigger condition to be met to trigger cell death of the feeder cells in the well 105. In other words, steps 908 and 909 achieve the effect of waiting until the timer has expired. When the timer has expired, step causes each of the feeder cells to be killed by controlling the one or more factors to cause the trigger condition, in order to leave only sample cells derived from the original sample cell remaining alive in the well 105.

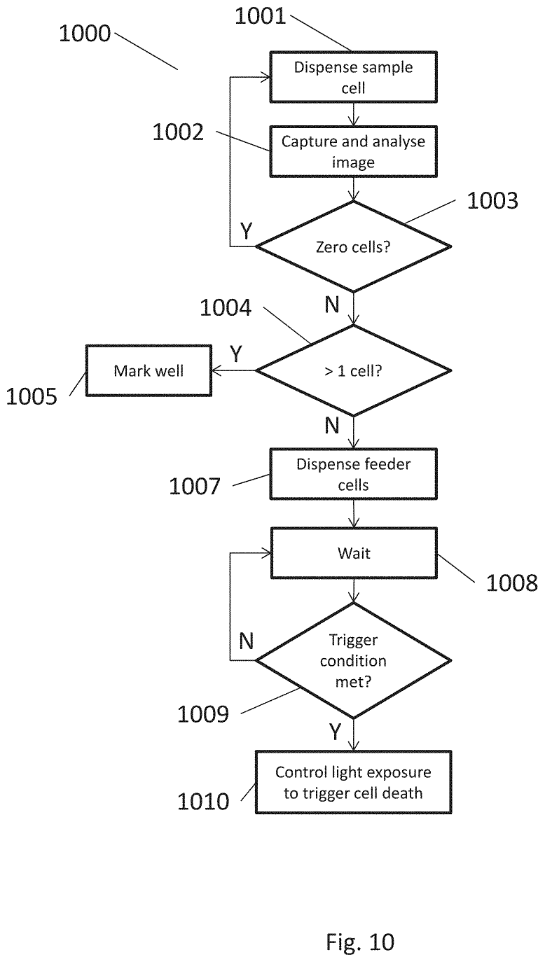

FIG. 10 illustrates a flow chart 1000 that shows a method of dispensing. In step 1001, a sample is dispensed into the current well 105. In step 1002, an image of the sample cell in the well 105 is captured and analysed. In step 1003 it is determined whether the receptacle (current well) 105 contains exactly zero cells, on the basis of the analysis of the captured image. If it does contain zero cells then the method returns to step 1001 to dispense a further sample cell into the well 105. However, if the well 105 contains more than zero sample cells, the method proceeds to step 1004 where it is determined whether the well contains more than one sample cell. If the well 105 does contain more than one sample cell, then the well is marked at step 1005 and an indication of the well may be stored in the storage unit 180. However, if the well 105 does not contain more than one sample cell it is concluded that the well 105 contains exactly one sample cell, therefore the method can proceed to step 1007 at which point at least one feeder cell is dispensed into the well 105 along with the sample cell. Then, at step 1008 the method waits (pauses) and at step 1009 it is determined whether or not the trigger condition to trigger cell death of the feeder cells is met. If the trigger condition is not met, the method returns to wait at step 1008, however if the trigger condition is met, then the method proceeds to step 1010 at which point cell death of the feeder cells is triggered by the light exposure of the receptacle being controlled. In other words, steps 1008 and 1009 achieve the effect of waiting until the trigger condition is met. When the trigger condition is met, step 1010 causes the light exposure of the receptacle to be controlled to trigger cell death of each of the feeder cells in order to leave only sample cells derived from the original sample cell remaining alive in the well 105.

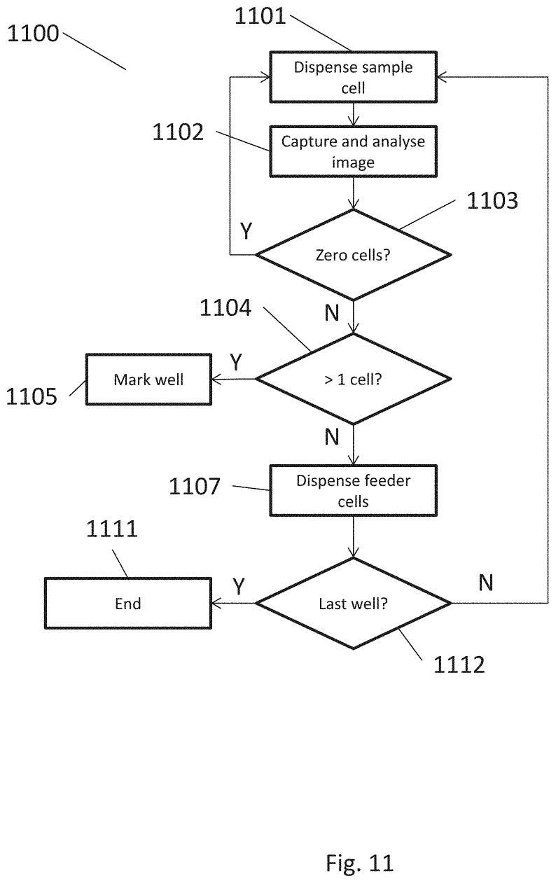

FIG. 11 illustrates a flow chart 1100 that shows a method of dispensing. In step 1101, a sample is dispensed into the current well 105. In step 1102, an image of the sample cell in the well 105 is captured and analysed. In step 1103 it is determined whether the receptacle (current well) 105 contains exactly zero cells, on the basis of the analysis of the captured image. If it does contain zero cells then the method returns to step 1101 to dispense a further sample cell into the well 105. However, if the well 105 contains more than zero sample cells, the method proceeds to step 1104 where it is determined whether the well contains more than one sample cell. If the well 105 does contain more than one sample cell, then the well is marked at step 1105 and an indication of the well may be stored in the storage unit 180. However, if the well 105 does not contain more than one sample cell it is concluded that the well 105 contains exactly one sample cell, therefore the method can proceed to step 1107 at which point at least one feeder cell is dispensed into the well 105 along with the sample cell. Then, at step 1112, it is determined whether or not the current well 105 is the last well in the microtiter well plate 110. If so, then at step 450, the process ends. Otherwise, at step 455, the microtiter well plate 110 is moved so that subsequent samples are dispensed into a different well. The process then returns to step 405 where a sample is dispensed.

In the present application, the words “configured to . . . ” or “arranged to” are used to mean that an element of an apparatus has a configuration able to carry out the defined operation. In this context, a “configuration” means an arrangement or manner of interconnection of hardware or software. For example, the apparatus may have dedicated hardware which provides the defined operation, or a processor or other processing device may be programmed to perform the function. “Configured to” or “arranged to” does not imply that the apparatus element needs to be changed in any way in order to provide the defined operation.

Although illustrative embodiments have been described in detail herein with reference to the accompanying drawings, it is to be understood that the invention is not limited to those precise embodiments, and that various changes, additions and modifications can be effected therein by one skilled in the art without departing from the scope of the invention as defined by the appended claims. For example, various combinations of the features of the dependent claims could be made with the features of the independent claims without departing from the scope of the present invention.

What is claimed is:

1. An apparatus comprising:
a determination unit to perform a determination as to whether a receptacle contains exactly one sample cell; and
a dispenser to dispense one or more feeder cells into the receptacle based on the determination,
wherein each of the one or more feeder cells is adapted to be responsive to a trigger condition being met to trigger death of that feeder cell.

2. The apparatus of claim 1, wherein
the dispenser is adapted to dispense the one or more feeder cells when the determination unit determines that the receptacle contains exactly one sample cell.

3. The apparatus of claim 1, further comprising:
a control unit to control one or more factors in order to cause the trigger condition.

4. The apparatus of claim 3, wherein
the one or more factors comprise an environment of the receptacle.

5. The apparatus of claim 3, wherein
the control unit is adapted to control the one or more factors when the sample cell has multiplied a predetermined number of times.

6. The apparatus of claim 5, wherein
the determination unit comprises an image capture device to capture an image of the receptacle to determine whether the sample cell has multiplied the predetermined number of times.

7. The apparatus of claim 3, wherein
the control unit is adapted to control the one or more factors after a predetermined period.

8. The apparatus of claim 4, wherein
the environment comprises a light exposure.

9. The apparatus of claim 4, wherein
the sample cell is resilient to the environment.

10. The apparatus of claim 1, wherein
the trigger condition is that the feeder cell has lived for a predetermined lifetime.

11. The apparatus of claim 1, further comprising:
a feeder cell reservoir to store feeder cells to be dispensed by the dispenser.

12. The apparatus of claim 1, wherein
the determination unit comprises an image capture device to capture an image of the sample in the receptacle to determine whether the receptacle contains only one sample cell.

13. The apparatus of claim 1, wherein
the dispenser is adapted to dispense the sample cell into the receptacle.

14. The apparatus of claim 1, wherein
the dispenser is adapted to dispense the sample cell into the receptacle when the determination unit determines that the receptacle contains exactly zero sample cells.

15. The apparatus of claim 1, further comprising:
a storage unit to store an indicator that the receptacle contains more than one sample cell when the determination unit determines that the receptacle contains more than one sample cell.

16. The apparatus of claim 1, wherein
the sample cell is a human tissue cell.

17. A method comprising the steps of:
performing a determination as to whether a receptacle contains exactly one sample cell; and
dispensing one or more feeder cells into the receptacle based on the determination,
wherein each of the one or more feeder cells is adapted to be responsive to a trigger condition being met to trigger death of that feeder cell.

18. An apparatus comprising:
means for performing a determination as to whether a receptacle contains exactly one sample cell; and
means for dispensing one or more feeder cells into the receptacle based on the determination,
wherein each of the one or more feeder cells is adapted to be responsive to a trigger condition being met to trigger death of that feeder cell.
